(19) United States
(12) Patent Application Publication
(10) Pub. No.: US 2024/0210389 A1
(43) Pub. Date: Jun. 27, 2024

(54) THREE-DIMENSIONAL PERCOLATION ARRAY STRUCTURE AS A GAS SENSOR

(71) Applicant: University of Utah Research Foundation, Salt Lake City, UT (US)

(72) Inventors: Hanseup KIM, Salt Lake City, UT (US); Farhan SIUM, Salt Lake Cityq, UT (US); Seungbeom NOH, Salt Lake City, UT (US); Carlos MASTRANGELO, Salt Lake City, UT (US)

(21) Appl. No.: 18/537,497

(22) Filed: Dec. 12, 2023

Related U.S. Application Data
(60) Provisional application No. 63/432,019, filed on Dec. 12, 2022.

(51) Int. Cl. G01N 33/543; B01L 3/00; G01N 33/00

(52) U.S. Cl. CPC G01N 33/5438; B01L 3/502715; G01N 33/0027; B01L 2300/0645; B01L 2300/0874

(57) ABSTRACT

An example three-dimensional percolation array includes a three-dimensional array of binding sites. The individual binding sites can be configured to selectively bind a molecule of a target chemical compound, thereby forming an electrically conductive connection between the electrically conductive structures. At least a portion of the binding sites can be electrically connected to at least one other binding site in a longitudinal direction, and to at least one other binding site in a lateral direction, and to at least one other binding site in a vertical direction.

### CROSS REFERENCE TO RELATED APPLICATIONS

This application claims priority to U.S. Provisional Patent Application No. 63/432,019, filed on Dec. 12, 2022, which is incorporated herein by reference.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION BY REFERENCE STATEMENT

Not applicable.

### BACKGROUND

Environmental gas detection and monitoring can be useful in a variety of applications. For example, regulations can require monitoring of certain industrial gases such as NO₂, SO₂, and CO. In agriculture it can be useful to monitor NH₃ and volatile organic compounds (VOCs). Oceanic life cycle gases such as CO₂, CH₄, and Oz can be monitored to provide information about oceanic life. Other gases can also be monitored in a variety of other fields. However, many types of gas sensors consume substantial amounts of power and include bulky or complex components, such as heaters, light sources and light focusing equipment, mechanical vibrators, vacuum pumps, and others. The high power consumption of these sensor can make them unsuitable for use in stand-alone, long-lasting sensor modules.

### SUMMARY

A three-dimensional percolation array can include a three-dimensional array of binding sites. The individual binding sites can comprise a nanogap between electrically conductive structures. The individual binding sites can be configured to selectively bind a molecule of a target chemical compound, thereby forming an electrically conductive connection between the electrically conductive structures. At least a portion of the binding sites can be electrically connected to at least one other binding site in a longitudinal direction, and to at least one other binding site in a lateral direction, and to at least one other binding site in a vertical direction.

An example chemically selective percolation switch can include a positive electrode, a negative electrode separated from the positive electrode by a horizontal switch gap; and a plurality of electrically conductive structures in the horizontal switch gap Adjacent electrically conductive structures can be separated by nanogaps forming binding sites between the adjacent electrically conductive structures. The conductive structures can be arranged to form a three-dimensional array of binding sites having at least two stacked layers of binding sites. The binding sites can be distributed in the switch gap such that the binding sites are capable of binding molecules of a target chemical compound to form an electrically conductive pathway via percolation between the positive electrode and the negative electrode when the chemically-selective percolation switch is exposed to a threshold concentration of the target chemical compound. The electrically conductive pathway can be capable of forming in one of the layers of binding sites or in more than one of the layers of binding sites.

An example digital chemical analyzer can include a power supply, a chemically selective percolation switch connected to the power supply, and a detection circuit connected to the chemically selective percolation switch to output a signal based on a change in at least one of resistance and current in the chemically selective percolation switch. The chemically selective percolation switch can include a positive electrode, a negative electrode separated from the positive electrode by a horizontal switch gap, and a plurality of electrically conductive structures in the horizontal switch gap. The power supply can be configured to apply a voltage between the positive electrode and the negative electrode. In the horizontal switch gap, adjacent electrically conductive structures can be separated by nanogaps forming binding sites between the adjacent electrically conductive structures. The electrically conductive structures can be arranged to form a three-dimensional array of binding sites having at least two stacked layers of binding sites. The binding sites can be distributed in the switch gap such that the binding sites are capable of binding molecules of the target chemical compound to form an electrically conductive pathway via percolation between the positive electrode and the negative electrode when the chemically selective percolation switch is exposed to a threshold concentration of a target chemical compound. The electrically conductive pathway is capable of forming in one of the layers of binding sites or in more than one of the layers of binding sites.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIGS. 1A-1B are schematic perspective views of an example three-dimensional percolation array in accordance with the present disclosure.

FIG. 2A is a side view of an example chemically sensitive percolation switch in accordance with the present disclosure.

FIG. 2B is a top-down view of the example chemically sensitive percolation switch in accordance with the present disclosure.

FIG. 2C is a perspective view of the example chemically sensitive percolation switch in accordance with the present disclosure.

FIG. 3 is a schematic side top view of an example percolation switch in accordance with the present disclosure.

FIG. 4 is a schematic side view of an example binding site in accordance with the present disclosure.

FIGS. 5A-SF show an example process for fabricating a three-dimensional percolation array in accordance with the present technology.

FIG. 6 is a schematic view of an example digital chemical analyzer in accordance with the present technology.

FIG. 7 shows graphs including I (current) vs. V (voltage) and the on/off response ratio of a three-dimensional percolation array in accordance with the present disclosure.

FIG. 8 shows the average bias voltage of the sensors having different dimensions.

FIG. 9 shows graphs of resistance (ohms) vs. time (seconds) and power (nW) vs. time.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “an electrode” includes reference to one or more of such features and reference to “the distributing” refers to one or more of such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “percolation” and “chemical percolation” refer to the natural phenomenon of molecules of a target chemical compound forming an electrical connection between two electrodes when the target chemical compound is present above a certain threshold concentration. This phenomenon is described in more detail below. As described in more detail below, electrically conductive structures can be placed to form an array of binding sites for the target chemical compound. Molecules of the target chemical compound can occupy binding sites and a continuous electrical connection can form across the array when the target chemical compound is present above the threshold concentration.

As used herein, “switch gap” refers to a gap between a positive electrode and a negative electrode of a chemically-selective percolation switch. In some examples, the switch gap can contain electrically conductive structures with binding sites between the electrically conductive structures.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Three Dimensional Percolation Arrays

As mentioned above, many existing gas sensors consume too much powder to be useful as a long-lasting, standalone gas sensor module. Despite recent rises in demand for real-time, battery-free gas monitoring systems for a variety of different gases, such gas sensor systems have been missing due to the mismatch between power consumption by the gas sensor and power generation by energy harvesters such as photoelectric cells. Power consumption of some existing gas sensors reaches more than 1 mW up to 500 mW due to heating, focused lights, mechanical vibration, or vacuum to achieve the desired selectivity or sensitivity in gas sensing. On the other hand, power generation by available energy harvesters, generally depending on the volume, remains at an average level of 0.1 mW/cm3 or <1 mW for a small sensor node on the order of 1 cm3.

One way of resolving the mismatch in power is to use an ultra-low-power gas sensor that consumes less than 10 nW power, similar to the leakage power level of recent electronics. Along these lines, a gas sensor was developed that included a single nanogap to capture a target gas molecule to form an electrically conductive path during gas detection. This sensor can remain in sleep mode when no target gas molecules are present, and in sleep mode the sensor can consume no power or very little power. When a target gas molecule forms an electrically conductive path across the nanogap the sensor can “wake up” and consume more power to register detection of the target gas. The average overall power consumption of the sensor can be less than 10 nW. This single gap sensor was further expanded into a 2D percolation array-based sensor to enhance reliability in gas detection. The 2D percolation array-based sensors are described in U.S. Pat. Nos. 10,502,724; 10,502,725 which are incorporated herein by reference. However, that 2D array structure and configuration required a higher voltage level of greater than 5 V, which is greater than the typical output value of a rechargeable battery. Therefore, the 2D percolation array-based sensors utilized a booster circuit, which increased power consumption of the sensors. As a result, the 2D percolation array-based gas sensors were insufficient to be integrated with a mainstream single-cell lithium-ion battery (which can produce 3.7 V) into a self-sustaining gas sensor node.

The present disclosure describes new gas sensors that utilize a three-dimensional percolation array. These sensors can operate at a lower voltage than the 2D percolation array-based sensors because the 3D percolation array provides multiple parallel possible conduction pathways in 3D. In some examples, the 3D percolation array can operate at a voltage lower than a single-cell lithium ion battery (e.g., less than 3.7 V). This can allow consistent and straightforward integration through a battery to various energy harvesters to realize a self-sustainable gas sensor node. This can also reduce the overall number of electronics in a sensor device and thus further reduce power consumption.

The sensors can also have increase tuning capabilities for designing a sensor with a desired percolation threshold. In some examples, the additional potential parallel pathways provided by the 3D array can allow the sensor to detect a target chemical compound at a lower threshold concentration. The sensors can also have better reliability and repeatability. The sensors can have lower false alarm rates compared to 2D array sensors.

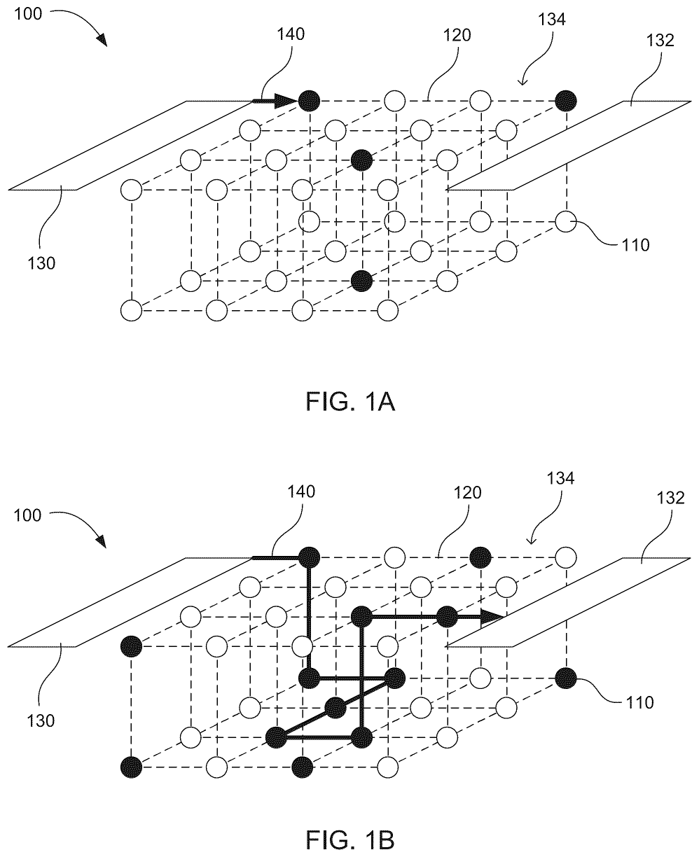

FIG. 1A is a schematic diagram of an example three-dimensional (3D) percolation array 100. This includes a 3D array of binding sites 110 The binding sites are represented as circles in this figure. The binding sites are locations where a molecule of a target chemical compound can be bound in a nanogap between two electrically conductive structures. However, the electrically conductive structure are not shown in this figure in order to clearly show the binding sites. The binding sites in this example are arranged in two 2D arrays of 4×4 binding sites. These 2D arrays are depicted in a perspective view as two stacked layers of binding sites. Thus, the 3D percolation array in this example is a 4×4×2 array. Each binding site can be capable of binding one or more molecules of a target chemical compound. In this example, binding sites that are occupied by a target molecule are filled in black, while binding sites that are unoccupied are filled in white. When a binding site is occupied by a target molecule, the target molecule can allow electric current to pass across the binding site. The unoccupied binding sites can be more electrically insulating because there is empty space in the binding site instead of a target molecule. The binding sites are also electrically connected to adjacent binding sites because current can flow through the electrically conductive structures. Although the electrically conductive structures are not shown in this figure, dashed lines are used to represent the electrical connections 120 between adjacent binding sites within each 2D array layer. Each binding site can be electrically connected to the binding sites next to it within the same 2D layer. Additionally, each binding site can be electrically connected to the binding site either directly below or directly above itself. Therefore, electric current can pass from the top 2D layer to the bottom 2D layer and vice versa.

The three-dimensional percolation array 100 shown in FIG. 1A also includes a positive electrode 130 and a negative electrode 132. The combination of the electrodes with the array of binding sites can also be referred to as a percolation switch. The space between the electrodes can be referred to as a horizontal switch gap 134, and the array of binding sites can be within this horizontal switch gap. In this example, the electrodes are electrically connected to the top layer of binding sites 110. However, in other examples, the electrodes can be electrically connected to the bottom layer of binding sites or to both layers of binding sites. The three-dimensional percolation array can conduct an electric current from one electrode to the other if a sufficient number of the binding sites are occupied and if the occupied binding sites are arranged to form a conductive pathway from one electrode to the other. In FIG. 1A, only a few of the binding sites are occupied. A black arrow is used to represent a conductive pathway 140 from the positive electrode to the negative electrode. In this figure, the electrically conductive pathway does not reach all the way to the negative electrode. Instead, the conductive pathway reaches only one of the binding sites adjacent to the positive electrode. At this point, there are no more adjacent occupied binding sites and the conductive pathway cannot continue.

FIG. 1B shows the same three-dimensional percolation array 100 with a greater number of occupied binding sites 110. In this example, an electrically conductive pathway 140 has formed between the positive electrode 130 and the negative electrode 132. The black arrow is used to represent the conductive pathway. In this example, the conductive pathway jumps from the top 2D layer of binding sites to the bottom 2D layer and then back again to the top 2D layer, so that a portion of the pathway is in the top layer and a portion is in the bottom layer. In other examples, it is possible that occupied binding sites might form a conductive pathway that is fully within the top 2D layer of binding sites. The three-dimensional array (made up of multiple 2D layers of binding sites) can provide more possibilities for the formation of electrically conductive pathways, and thus make it easier for electrically conductive pathways to form across the horizontal switch gap 134 between the electrodes.

The arrays of binding sites described herein are referred to as “percolation” arrays because they can operate through the phenomenon of chemical percolation. When target chemical molecules become sufficiently available, the molecules will statistically adhere to the binding sites between the electrodes and form continuous paths for electron tunneling. However, a continuous path across the entire array will occur only when the concentration of the target chemical is above a certain threshold. This concentration can be referred to as the percolation threshold.

In some examples, a bias voltage can be applied across the electrodes, and the target chemical compound can be detected based on a measured property such as the resistivity of the percolation array or the amount of electric current flowing between the electrodes. If the concentration of the target chemical compound is lower than the percolation threshold, then the electric current flowing between the electrodes will be zero or relatively low. If the concentration of the target chemical compound is higher than the percolation threshold, then the electric current will be greater. In some cases, a sharp increase in the electric current can be observed when the concentration of the target chemical compound crosses the percolation threshold. As mentioned above, using a three-dimensional percolation array instead of a two-dimensional percolation array can allow for a lower bias voltage to be used while detecting the target chemical compound. Having a 3D array of binding sites can allow for more possible conductive pathways to form between the electrodes compared to a 2D array.

It is noted that all physical objects are three-dimensional. Thus, the term “2D array” is not intended to refer to some sort of two-dimensional object. Rather, the term refers to an array of binding sites that can be represented as a two-dimensional array. In the arrays of binding sites described herein, at least a portion of the binding sites can be electrically connected to at least one other binding site. In a 2D array, the binding sites and their connections can all be drawn conceptually in a single 2D plane. Of course, in practice a 2D array of binding can be made from three-dimensional materials, such as electrically conductive metal layers that have length, width, and height dimensions. In some cases, the binding sites in such an array may be positioned in a way that the binding sites are not all positioned in a single plane. However, if the binding sites and their electrical connections can be conceptually drawn as being in a single 2D plane, then the array can be referred to as a 2D array.

A 3D array of binding sites can be different from a 2D array in that the binding sites and their connections cannot all be in a single 2D plane. As shown in FIGS. 1A and 1B, a 3D array can be made up of multiple stacked 2D array layers. At least some of the binding sites in one of the stacked 2D array layers can be electrically connected to binding sites in the other stacked 2D array layer. In certain examples, each binding site can be electrically connected to a binding site that is directly above itself or directly beneath itself, in the other 2D array layer. In further examples, the 2D array layers may be offset so that the binding sites do not line up one above another, but the binding sites can still be electrically connected to one or more other binding sites in the other 2D array layer.

In the 3D array of binding sites, at least a portion of the binding sites can be electrically connected to at least one other binding site in a longitudinal direction, and to at least one other binding site in a lateral direction, and to at least one other binding site in a vertical direction. The longitudinal direction can refer to the “end-to-end” direction of an array. In some cases, this can be the direction from one electrode to another across the array. The lateral direction can refer to the “side-to-side” direction, which can be substantially orthogonal to the longitudinal direction. The vertical direction can refer to the “up-and-down” direction. Stating that a binding site is connected to another binding site in the vertical direction can refer to a binding site in one stacked 2D array layer being connected to a binding site in a different stacked 2D array layer. It is noted that the binding sites are not necessarily arranged in a strict grid along the longitudinal axis, lateral axis, and vertical axis. For example, binding sites can be arranged in a triangular grid, a hexagonal grid, or some other arrangement. Furthermore, stacked 2D array layers are not necessarily stacked with binding sites directly one on top of another. In some examples, stacked 2D array layers can be offset by any distance. In some examples, binding sites can be electrically connected to other binding sites that are positioned at some distance along multiple axes, such as along the longitudinal axis and along the lateral axis, or along the longitudinal axis and along the vertical axis. Therefore, the arrays described herein are not limited to strict square grid patterns or rectangular grid patterns.

The number of binding sites in the 3D array can vary. In some examples, the 3D array can have dimensions from 4×4×2 to 100×100×100 (referring to the number of binding sites in the longitudinal, lateral, and vertical directions, respectively). In certain examples, the three dimensional array can be made up of multiple stacked 2D arrays of binding sites. The number of stacked 2D arrays can be from 2 to 100 in some examples, or from 2 to 50, or from 2 to 30, or from 2 to 20, or from 2 to 10, or from 2 to 5, or from 5 to 10, or from 5 to 20, or from 5 to 50 in other examples. The 2D arrays can have from 4 to 100 binding sites in the longitudinal and/or lateral directions in some examples. In further examples, the 2D arrays can have from 4 to 64, or from 4 to 36, or from 4 to 25, or from 4 to 16, or from 16 to 64, or from 25 to 64 binding sites in the longitudinal and/or lateral directions.

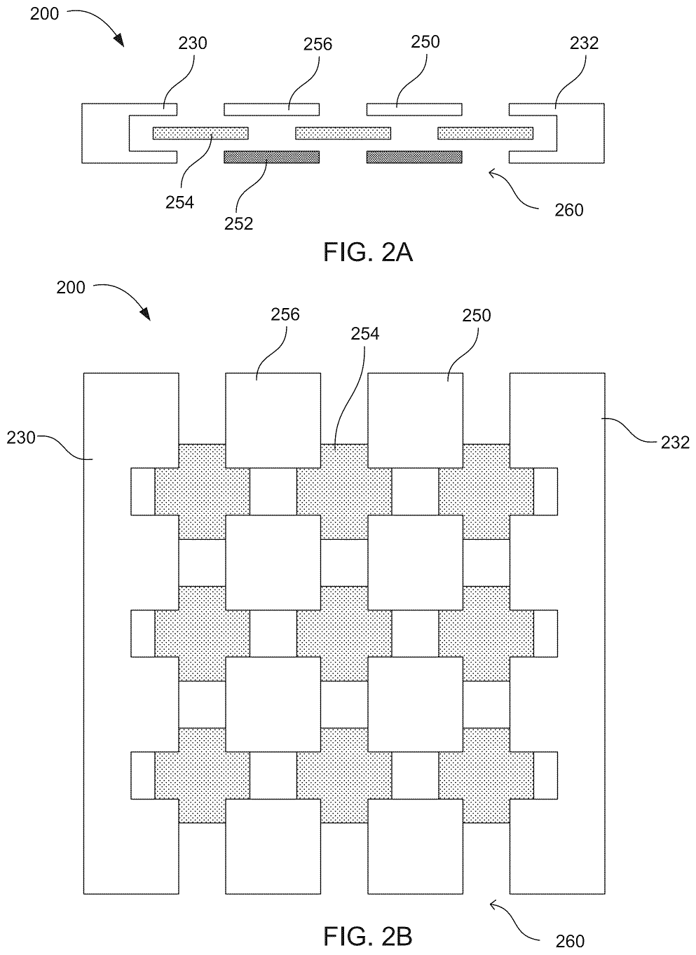

As mentioned above, the binding sites can be formed as nanogaps between electrically conductive structures. These electrically conductive structures can be designed with a variety of shapes, sizes, and arrangements as long as there are gaps between the structures that can accommodate molecules of the target chemical compound. In some examples, the electrically conductive structures can be horizontal parallel plates. These can be made of a conductive material such as metal. FIG. 2A is a side view of an example percolation switch 200 that includes a plurality of electrically conductive structures 250 in a horizontal switch gap 260 between a positive electrode 230 and a negative electrode 232. The electrically conductive structures are square-shaped horizontal metal plates in this example. The plates are arranged in stacked horizontal layers, including a bottom layer 252, a middle layer 254, and a top layer 256. The adjacent layers are offset so that the corners of the square plates overlap FIG. 2B shows a top-down view of the percolation switch, showing how each metal plate in the top layer overlaps at the corners with the corners of four other metal plates in the middle layer (with the exception of the plates at the edges of the array, which overlap with only two plates in the middle layer). The bottom layer of metal plates is aligned directly under the metal plates of the top layer, and therefore the bottom layer is not visible in the top-down view. FIG. 2C shows a perspective view of this percolation switch. In this figure, all three of the layers of overlapping metal plates can be seen. In this example, the binding sites are located in the nanogaps between the metal plates in the overlapping areas where the corners overlap. When one or more molecules of a target chemical compound are bound in a nanogap, then an electrically conductive pathway is formed between two plates. In this design, the metal plates in the middle row can be electrically connected to up to eight other metal plates: four overlapping metal plates in the top row and four overlapping metal plates in the bottom row. Each of these eight potential connections is a binding site that may be occupied by a molecule of the target chemical composition. The 3D array of binding sites in this example has dimensions of 6×6×2. In these figures, the metal plates are depicted as floating in mid-air. In practice, the metal plates can be supported by support material that is non-conductive, such as silicon or other materials.

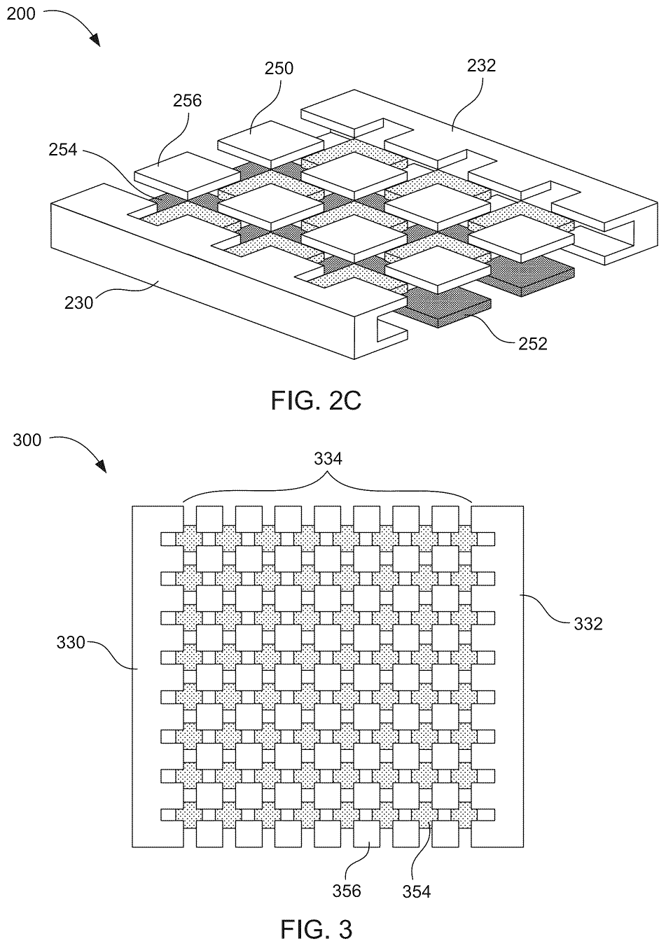

Another example percolation switch 300 is shown in FIG. 3. This figure shows a top-down view of a chemically selective percolation switch that has a positive electrode 330 and a negative electrode 332 separated by a horizontal switch gap 334. An array of binding sites is formed in the switch gap. The binding sites are located between overlapping portions of electrically conductive horizontal parallel plates. This example has three layers of horizontal parallel plates: a top layer 356, a middle layer 354, and a bottom layer that is not visible in the top-down view. In this example, the horizontal plates are square-shaped and arranged in a square grid pattern. The top layer has dimensions of 7 plates in the longitudinal direction (i.e., the electrode-to-electrode direction) and 9 plates in the lateral direction. The electrodes are also formed with fingers having the same width as the horizontal plates, so that the fingers partially overlap with the horizontal plates to form binding sites as well. The middle layer of horizontal plates has dimensions of 8 plates in the longitudinal direction by 8 plates in the lateral direction. Binding sites are located at each overlapping portion between overlapping plates. With the three layers of horizontal plates arrange in the way shown in this example, the overlapping portions form a three-dimensional array of binding sites with dimensions of 16 binding sites in the longitudinal direction by 16 binding sites in the lateral direction by 2 binding sites in the vertical direction (16×16×2).

In other examples, the chemically selective percolation switch can be designed with a different number of electrically conductive horizontal plates in the horizontal switch gap. The number of horizontal plates can be from 2 to 50 in the longitudinal and/or lateral directions in some examples, or from 2 to 40, or from 2 to 32, or from 2 to 18, or from 2 to 13, or from 2 to 8, or from 4 to 50, or from 4 to 32, or from 4 to 16 in the longitudinal and/or lateral directions. In the vertical direction, the number of layers of horizontal plates can be from 3 to 101 in some examples, or from 3 to 65, or from 3 to 37, or from 3 to 26, or from 3 to 17, or from 3 to 10 in other examples.

The dimensions of the individual electrically conductive horizontal plates is not particularly limited. In some examples, the horizontal plates can have a width and length that is small to allow for the entire switch design to be compact. In certain examples, the horizontal plates can have a length and/or width from 1 μm to 1 mm, or from 1 μm to 500 μm, or from 1 μm to 200 μm, or from 1 μm to 100 μm, or from 100 μm to 1 mm, or from 200 μm to 1 mm, or from 500 μm to 1 mm. The thickness of the plates is also not particularly limited, but in some examples the thickness can be from 0.1 nm to 100 μm, or from 0.1 nm to 10 μm, or from 0.1 nm to 1 μm, or from 0.1 nm to 500 nm, or from 0.1 nm to 100 nm, or from 0.1 nm to 50 nm, or from 100 nm to 1 μm, or from 100 nm to 500 nm. In some cases, the thickness of the plates can depend on the process used to form the plates. Example deposition processes that can be used to form the horizontal plates are described in more detail below.

In the example shown in FIG. 3, each of the electrically conductive horizontal plates overlaps partially with other horizontal plates. The plates in the middle layer of plates overlap partially with 8 other plates: 4 plates in the top layer and 4 plates in the bottom layer, with the exception of the middle-layer plates nearest to the electrodes. The middle-layer plates next the electrodes overlap with 2 top-layer plates and 2 bottom-layer plates, and then the other two corners of the middle-layer plates overlap with fingers of the electrode. Other designs can include electrically conductive structures that have a different shape and which form nanogaps with a different number of other electrically conductive structures. In various examples, at least some of the electrically conductive structures can form nanogaps with 2 to 8 other electrically conductive structures. In other words, when the target chemical compound is present and occupies all the available binding sites, the electrically conductive structures can be connected to 2 to 8 other electrically conductive structures. The binding sites are electrically connected one to another through the electrically conductive structures. In the example of FIG. 3, the binding sites are each directly electrically connected to 10 other binding sites through the two horizontal plates on either side of the binding site. As an example, a binding site in the top layer of the 3D array of binding sites is located between a corner of a middle-layer horizontal plate overlapping with a corner of a top-level horizontal plate. The middle-layer plate connects this binding site directly to the binding sites at the other 3 corners on the top surface of the middle-layer plate. The middle-layer plate also directly connects the binding site to the 4 binding sites under the middle-layer plate, which are in the bottom layer of the 3D array of binding sites. On the other side of the nanogap, the top-layer plate directly connects the binding site to the other 3 binding sites on the bottom surface of the top-layer plate. Thus, the binding site is directly electrically connected to a total of 10 other binding sites. If additional layers of horizontal plates were added to this design, then the binding sites in the interior layers would be directly connected to 14 other binding sites. In various examples, at least a portion of the binding sites can be directly electrically connected to 3 to 14 other binding sites, depending on the design of the electrically conductive structures.

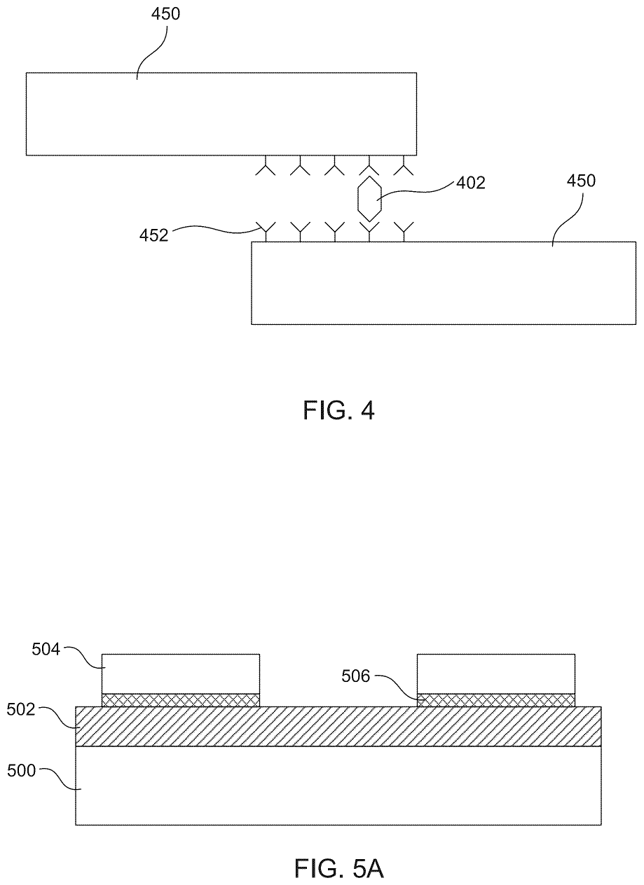

A closer view of an example binding site is shown in FIG. 4. This figure shows a side view of an overlapping portion of two horizontal electrically conductive plates 450. The gap between the plates can be a nanogap (i.e., a gap with a gap width on the scale of nanometers). In some examples, the nanogap can have a gap width from 0.3 nm to 1,000 nm. This nanogap between the plates is also referred to as a binding site, because this is the location where one or more molecules of a target chemical compound 402 can be bound to form an electrically conductive connection between the plates. In this example, a binding agent 452 has been attached to the surface of the horizontal plates on both sides of the nanogap. The binding agent can be a chemical compound that can selectively bind to the target chemical compound.

This type of binding site can be highly selective for binding the target chemical compound because it utilizes two mechanisms for selectivity: chemical selectivity and size selectivity. Chemical selectivity is provided by the binding agent, which can include one or more functional groups that selectively bind to the target chemical compound. Size selectivity can be provided by selecting a nanogap distance that accommodates a single molecule of the target chemical compound. Other compounds that are larger than the target compound can be excluded because they will not fit into the nanogap, and smaller compounds will not fully bridge the nanogap and therefore will not affect the electrical conductivity in the same way that the target chemical compound will.

Various target chemical compounds can fit in differently sized nanogaps. In various examples, the nanogap can have a gap distance from 0.3 nanometer to 1,000 nanometers, or from 0.3 nanometer to 500 nanometers, or from 1 nanometer to 300 nanometers, or from 1 nanometer to 200 nanometers, or from 1 nanometer to 100 nanometers, or from 1 nanometer to 50 nanometers, or from 1 nanometer to 20 nanometers. In some examples, the binding site can utilize size-based exclusion alone to provide selectivity, in which case no binding agent is used. In these examples, the gap distance can be selected to match as closely as possible the length of a molecule of the target chemical compound.

In other examples, a binding agent can be attached to the electrically conductive structures on one or both sides of the nanogap. The binding agent can be a compound that can be immobilized on surfaces of the electrically conductive structures. The binding agent can also be capable of reversibly binding with the target chemical compound. In some examples, the binding agent can selectively bind the target chemical compound through hydrogen bonding. In certain examples, the binding agent can bind to the target chemical compound through ultra-selective host-guest recognition. This host-guest recognition is the process of holding molecules without covalent (permanent) bonding. A target molecule is adsorbed by a host molecule, such as a crown ether, when the size, shape and charge-distribution of the target and the host match with each other, leading to ultra-specific binding. Since this does not form covalent bonding, the binding can be breakable, and the binding agent thermo-dynamically desorbs the target molecules to reach a lower Gibbs energy equilibrium, enabling reversibility of adsorption. The reversibility depends on adsorption process (instead of absorption) where the target molecules temporarily attach onto the binding sites. As the adsorption period becomes longer, then the reversibility time becomes longer. The length, charges, etc. of the binding agent can be selected to tune the half-adsorption-lifetime of the receptor complex. In certain examples, the electrically conductive structures of the sensor can be treated with amine-PEG-amine and crown-tetracarboxylic-acid layers, forming binding sites for target molecules. In further examples, the binding agent can link to the surfaces of electrically conductive structures through a linking group such as a thiol group, an amine group, a siloxy group, or another linking group.

The selection of binding agents can also affect the threshold concentration at which a continuous conductive pathway will form across the switch. Binding agents can be selected to have a high degree of conjugation to allow for more electrical conductivity. The length of the binding agent molecules can also be selected to make an appropriately sized space for a single target molecule to be captured between two binding agent molecules. The type of capture group on the binding agent molecule can be selected to match with the target molecule. For example, the capture group can include hydrogen bond donors spaced apart at a distance that matches with hydrogen bond acceptors on the target molecule.

In some examples, the binding agent can have a x-conjugated structure and can be electrically conductive. The binding agent can include a core group with a x-conjugated structure and a side binding group that can be modified without altering the electrical properties of the core group. In certain examples, the binding agent can include a sulfhydryl (—SH) group, which can be used to link the binding agent molecule to the electrically conductive structures. The binding agent may also include a second sulfhydryl group to bind to the target chemical compound.

In some examples, the binding agent molecule can have a molecule length from about 1 nm to about 5 nm, or from about 2 nm to about 3 nm. Non-limiting examples of the binding agent can include conductive molecules custom-designed or commercially available, such as, but not limited to, thiol-functional-group to bind hexanal-functional-group chemicals, aldehyde- or hydroxyl-functional-group to bind carboxyl-functional-group chemicals, hydrophobic interaction to bind the alkane-functional-group chemicals, and hydrogen-bonding to bind ester-functional-group chemicals, in some examples. Note that these binding groups can be formed at the end of a backbone structure that can have multiple forms to maintain conductivity and rigidness.

The chemically selective percolation switches can be designed to detect a variety of target chemical compounds. In certain examples, the target chemical compound can include chemical warfare agents, including paralytic shellfish toxins (PST), such as saxitoxin, tetrodotoxin, zetekitoxin, chiriquitoxin, or sarin. In other examples, the target chemical compound can be a fuel, an air pollutant, an airborne compound, an explosive, an airborne biological agent, or combinations thereof. Other examples can include volatile organic compounds released by plants or animals. Some example volatile organic compounds released by plants include hexanal, hexenal, hexenol, acetaldehyde, decanal, diamine, ethylene, indole, terpene, acetone, pentanal, 4-methylpentan-2-one, toluene, and dibutyl phthalate. Some example volatile organic compounds released by animals include methanal 2-amino-5-isopropyl-8-methyl-1-azulenecarbonitrile, 3,3-dimethyl (Formaldehyde). pentane, 5-(2-methylpropyl)nonane, 2,3,4-trimethyl decane, 2-Trifluoromethylbenzoic acid, 6-ethyl-3-octyl ester, 2-Butanone, butanal, 2-pentanone, pentanal, hexanal, heptanal, octanal, acetone, Isobutane, 2,3,4-trimethyl hexane, 1-hexene, benzene, ethylbenzene, 1-methyl-4-(1-methylethyl)benzene, p-xylene, m-xylene, o-xylene, methanol, isopropanol, 1-propanol, butyraldehyde, Nonanal, isononane, isoprene, styrene, toluene, ethanol, 2-ethylhexanol, Decanal, Hexadecane, Undecanal, dodecanal, pentadecanal, cyclohexanone, 4-methylanisol, hexyl ethylphosphonofluoridate, indole, 2-pentylfuran, 6-ethyl-2 methyl Decane, Oxirane-dodecyl, 2,4,4-trimethyl-1-pentene, 1,3,5-tri-tert-butylbenzene, menthyl acetate, Butylated hydroxytoluene, Cyclohexanol, phenol, 2-propanol, Pentanoic acid, Butanoic acid, Benzofuran, Hydrogen nitrate, ethyl acetate, Methylthiocyanate, Hydrogen cyanide, 2-Aminoacetophenone, Propane, 2-methoxy-2-me, Cyclohexane, 1,3-dimethyl-, trans-Cyclohexane, Pentane, 1,4-dimethyl-Cyclohexane, 2,4-dimethyl-Heptane, 1-ethyl-4-methyl-, trans-Cyclohexane, 3-ethyl-2-methyl-Heptane, 2,6-dimethyl-Octane, 3-methyl-Heptane, 4-methyl-Heptane, 4-methyl-Decane, Tridecane, 1-_beta_-Pinene, Camphene, 3,6,6-trimethyl-Bicyclo_3_1_1_hept-2-ene, 1-Octene, methyl benzene, 1,4-dichloro benzene, 1,2,3,4-tetramethyl-Benzene, ethyl benzene, 1-methyl-naphthalene, 2-methyl-Styrene, propyl benzene, 2-butyl-1-octanol, Furfural, 6-methyl-5-hepten-2-one, 2-butoxy-ethanol, 2-propenenitrile, 2-Ethyl-1-hexanol, 5-Methyl-3-hexanone, 2,2-Dimethyl-propanoic acid, 4-(4-propylcyclohexyl)-4′-cyano[1,1′-biphenyl]-4-yl ester benzoic acid, 1,3-dimethyl benzene, 1,1′-(1-butenylidene)bis benzene, [(1,1-dimethylethyl)thio]acetic acid, 1-jodo nonane, Hydrogen sulfide, Methyl mercaptan (Methanethiol), Dimethyl sulfide, Dimethyl disulfide, Dimethyl trisulfide, Ammonia, Nitric oxide, ethane, methylene chloride, Bicyclo[2.2.1]heptane, 2,2,3-trimethyl-, exo-, 4,6-Dimethyl-dodecane, Limonene, 3-methylhexane, 5-ethyl-3-methyloctane, nonane, 2,2-dimethyl decane, Ethylene, 2,3-dihydro-benzofuran, acetic acid, methane-sulfonyl chloride, p-xylene, 3-carene, terpenes, α-pinene, Methyl Nitrate, 2-pentanone, and ethyl butanoate.

Regarding the construction of the 3D percolation arrays described herein, any suitable fabrication processes can be used to make the arrays described above. In some examples, the processes used can be capable of forming electrically conductive structures separated by nanogaps on the order of nanometers or less, such as 0.3 nm to 10 nm nanogaps. Therefore, a fabrication method can be selected that allows fine control over the nanogap distance. In certain examples, the arrays can be formed using microfabrication processes for depositing and patterning thin layers of material. Fabrication processes that can be used include sputtering, atomic layer deposition, physical vapor deposition, chemical vapor deposition, spin coating, lithography, etching, and others.

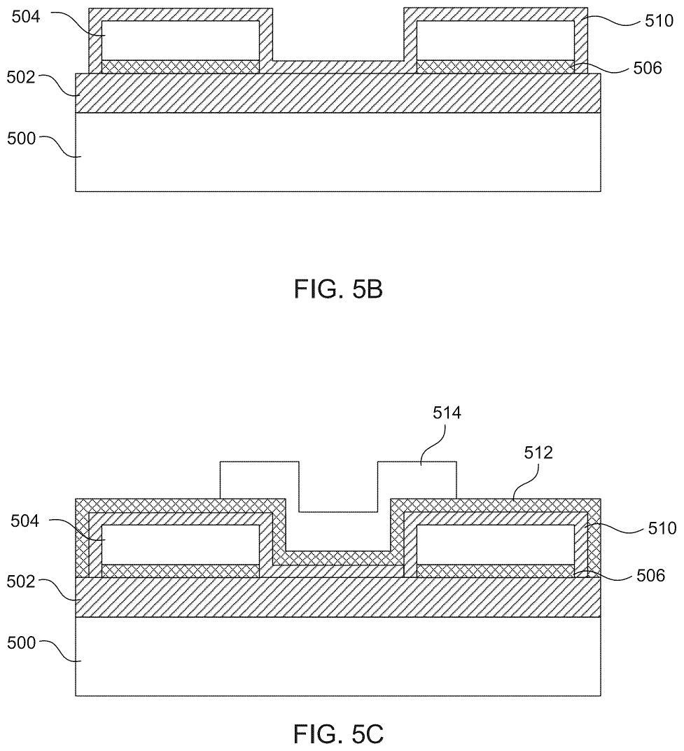

An example process for fabricating a 3D percolation array is shown in FIGS. 5A-5F. In FIG. 5A, a substrate 500 is prepared from a substrate material such as a silicon wafer or any other suitable substrate material. An insulating layer 502 is formed on the substrate. The insulating material can be an electrically insulating dielectric material such as SiO₂ to electrically isolate the substrate from the elements formed above. Two electrically conductive horizontal plates 504 were then formed with an adhesion layer 506 between the plates and the insulating layer. The electrically conductive horizontal plates can be made from a conductive material, such as a metal. Examples can include gold, copper, silver, graphite, titanium, or others. The horizontal plates can be formed with any desired shape using a suitable fabrication process such as lithography. In this example, the horizontal plates are square-shaped (but the square shape is not visible when viewed from a cross-sectional side view, as in this figure).

The adhesion layer can be used to increase adhesion of the electrically conductive horizontal plates to the insulating layer. Therefore, the adhesion layer can be made of a material that has good adhesion both to the insulating layer and to the electrically conductive material of the plates. In some examples, a chromium adhesion layer can be used with gold horizontal plates, or with plates made of other metals. Additional examples of adhesion layer materials can include tungsten, niobium, or titanium in other examples.

The thicknesses of the layers shown in FIG. 5A are not particularly limited because these are the bottom layers of the array, and the thicknesses of these layers will not affect the nanogap distance. In some examples, the insulating layer can have a thickness from 1 nm to 1,000 nm or more. In further examples, the initial adhesion layer can have a thickness from I nm to 50 nm. The electrically conductive horizontal plate thickness can be from 0.1 nm to 100 μm in some examples, and in certain examples the thickness can be from 10 nm to 500 nm, or from 100 nm to 500 nm.

FIG. 5B shows a second insulating layer 510 formed over the electrically conductive horizontal plates. This layer can be made of the same insulating material as the first insulating layer or a different insulating material. The thickness of the second insulating layer can be equal to or less than the desired nanogap distance. In some examples, the thickness of this layer can be from 0.1 nm to 1 μm, or from 0.3 nm to 500 nm, or from 1 nm to 100 nm, or from 1 nm to 10 nm, or from 1 nm to 5 nm.

FIG. 5C shows a second adhesion layer 512 and a second electrically conductive layer 514 formed over the second insulating layer 510. The second adhesion layer can also have a thickness that is less than the nanogap distance. In some examples, a portion of the second adhesion layer and the second insulating layer can be removed to form the nanogap, and therefore the combined thickness of the second adhesion layer and the second insulation layer will be equal to the nanogap distance. In some examples, the second adhesion layer can have a thickness from 0.1 nm to 1 μm, or from 0.3 nm to 500 nm, or from 1 nm to 100 nm, or from 1 nm to 10 nm, or from 1 nm to 5 nm. The second electrically conductive layer is deposited using one of the electrically conductive materials described above and patterns to form a horizontal plate. The horizontal plate can have the same shape as the horizontal plates on the bottom layer in some examples. In this example, the second electrically conductive layer is shaped as a square horizontal plate when viewed from above. The electrically conductive material is deposited conformally in this example, so that a depression forms in the center of the plate. This does not interfere with the operation of the sensor as long as the plate is continuous so that the plate can conduct electricity throughout the plate. The thickness of the second electrically conductive layer can be in the ranges described above for the electrically conductive horizontal plates on the bottom layer. When viewed from above, the horizontal plate of the second electrically conductive layer can overlap at its corners with corners of the plates in the bottom layer.

FIG. 5D shows a third insulating layer 520 deposited over the second electrically conductive layer. This insulating layer can have a thickness in the ranges described above for the second insulating layer. FIG. 5E shows a third adhesion layer 522 formed over the third insulating layer and a third electrically conductive layer formed over the third adhesion layer. The third adhesion layer can also have a thickness as described above for the second adhesion layer. The third electrically conductive layer 524 is patterned to form two horizontal plates, having a similar shape to the plates in the bottom layer. These plates can overlap at their corners with the corners of the plate of the second layer when viewed from above. The plates of the third layer can be made from the same materials and have a thickness in the ranges described above for the other electrically conductive plates.

FIG. 5F shows the array after an etching process has been used to remove portions of the insulating material and the adhesion material. In particular, the etching process removes a portion of the third adhesion layer 522 and the third insulating layer 520. Other portions of these layers remain underneath the electrically conductive plates on the top layer. However, the etchant is able to remove a certain portion of the insulting material and adhesion material from under the edges of the electrically conductive plates. Thus, an empty nanogap 530 is left between the electrically conductive plates in the top layer and the electrically conductive plate in the middle layer. The etchant can also reach a portion of the second insulating layer and the second adhesion layer. Although it is not visible in this cross section, there are locations in the array where no electrically conductive material has been deposited, and these locations have only layers of insulating material and adhesion material. Thus, in these areas, the etchant can remove the insulating material and the adhesion material all the way down to the substrate in some examples. Additionally, the etchant can reach some of the insulating and adhesion material under the edges of the electrically conductive plate in the middle layer. This forms a nanogap between the plate in the middle layer and the plates in the bottom layer. For convenience, these lower nanogaps 532 are illustrated in this figure even though they would be present in a different cross-section of the array. The etching process can utilize an etchant that can remove the insulating material and the adhesion material without removing the electrically conductive material. Alternatively, the etchant can remove the insulating material, adhesion material, and the electrically conductive material, but the insulating material and the adhesion material in the nanogaps can be removed more quickly than the electrically conductive material of the plates. In certain examples, wet etching, dry etching, reactive ion etching (RIE), and combinations thereof can be used. In further examples, an etching process for removing the adhesion material can be alternated with an etching process for removing the insulating material. In certain examples, the adhesion material can be chromium and the chromium can be etched with a wet chromium etchant, while the insulating material can be silicone dioxide which can be etched using RIE.

After the nanogaps have been formed, ambient air can be in contact with the nanogaps between all the layers of electrically conductive plates. Therefore, if a target chemical compound is present in the ambient air, molecules of the target chemical compound can diffuse or otherwise move to the surfaces of the electrically conductive plates in the nanogaps. The nanogaps can be formed with a specific gap distance that matches the length of the target chemical compound in some examples. The array can also be treated with a binding agent compound at this point. The binding agent can attach to the electrically conductive plates on the sides of the nanogap. In such examples, the gas distance of the nanogaps can provide room for the length of the target compound molecule plus the length of the binding agent molecules.

To construct arrays having additional layers of electrically conductive plates, the same steps of depositing an insulating layer, an adhesion layer, and a patterned electrically conductive layer can be repeated as many times as desired to increase the vertical dimension of the array. Additionally, electrodes can be formed of an electrically conductive material by patterning a layer of electrically conductive material in the same way that the electrically conductive plates are formed.

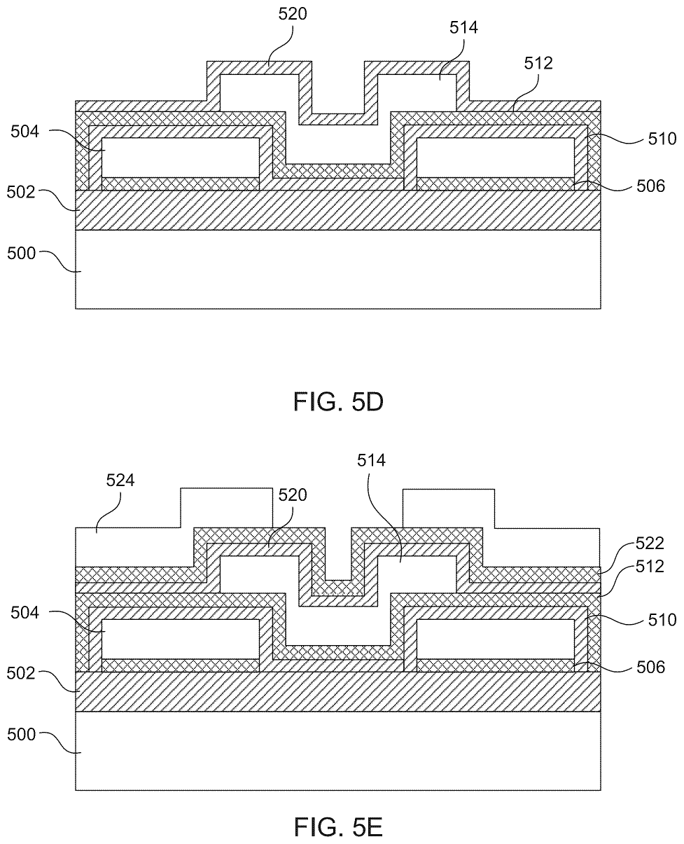

The chemically sensitive percolation switches described herein can be incorporated into devices for detecting a target chemical compound. In some examples, the device can be a digital chemical analyzer. FIG. 6 is a schematic diagram of an example digital chemical analyzer 600 including a power supply 610, a chemically selective percolation switch 620 connected to the power supply, and a detection circuit 630 connected to the chemically selective percolation switch. The detection circuit can output a signal based on a change in the resistance of the electric current in the chemically selective percolation switch. The chemically selective percolation switch can be in the “off” state when the target chemical compound is not present. In this state, there are no molecules of the target chemical compound in the nanogaps in the chemically selective percolation switch, and therefore the amount of current that flows through the switch is either zero or very low because of the high resistivity of the switch. If the target chemical compound is present at a concentration above the percolation threshold then the switch can switch to the “on” state, in which a sufficient number of molecules of the target chemical compound bind in the nanogaps to form a continuous electrically conductive pathway through the switch. When the switch is in the “on” state, a greater electric current can flow through the switch. This can be detected by the detection circuit, and the detection circuit can output a signal indicating that the switch is in the “on” state.

In some examples, the detection circuit can be as simple as a light emitting diode (LED), or alarm, or other signal device that can be powered by the power supply. The LED, alarm, or other signal device can be activated and powered by electric current that flows through the switch when the switch is in the “on” state. In other examples, more complex detection circuits can be used. In some examples, the detection circuit can include a processor, a microcontroller, a comparator, an amplifier, a battery, a display, a wireless communication module, or a combination thereof. In certain examples, the detection circuit can be programmed to remain in a “sleep” state until a sufficient electric current or voltage passes through the chemically selective percolation switch to wake the detection circuit.

The power supply used to power the chemically selective percolation switch can include an energy harvester, such as a solar panel or a wind-powered generator. In some examples, the power supply can also include a battery connected to the energy harvester. The battery can provide voltage to the chemically selective percolation switch, while the energy harvester can recharge the battery at times when wind or solar energy is available. In certain examples, the battery can be a single cell lithium ion or lithium polymer battery. The battery can have a nominal voltage of 3.7 V in some examples. The actual voltage supplied by the battery can vary between 3.2 V and 4.2 V in some examples. Other types of batteries can also be used, with a voltage in the range of about 1.4 V to about 4.2V. Some percolation switches that include 2D percolation arrays of binding sites have been found to run at a minimum voltage of 5 V or higher. Thus, batteries in the voltage range of 1.4 V to 4.2 V would not be sufficient to power such 2D percolation arrays. However, the 3D percolation arrays described herein have been found to be usable with lower voltages that can be achieved using a single cell battery.

The power supply can be used to apply a bias voltage from 1.4 V to 4.2 V across the chemically selective percolation switch. When the switch is in the “off” state (because the target chemical compound is present at a concentration below the threshold concentration), the power supply can operate at a power of less than 10 nW because the switch conducts no current or a very small current. In further examples, the power supply can operate at a power less than 5 nW or less than 1 nW when the switch is in the “off” state.

The digital chemical analyzer can operate in a low-power “sleep mode” when the target chemical compound is not present. As explained above, little or no electric current flows across the switch gap of the chemically selective percolation switch when the target chemical compound is not at the threshold concentration. Thus, no power is consumed by the positive and negative electrodes when the target chemical compound is not present. In some examples, the digital chemical analyzer can also include a battery and a microcontroller. The microcontroller can normally be shut off or in a low power sleep mode when the target chemical compound is not present. However, the microcontroller can include a “wake-up” circuit that turns on the microcontroller when the target chemical compound reaches the threshold concentration. For example, the wake-up circuit can be connected to the switch gap. When molecules of the target chemical compound bridge the switch gap, electric current can flow across the switch gap. However, the magnitude of the electric current can be fairly small compared to the electric current normally used to run the microcontroller. The wake-up circuit can be triggered by the small current from the switch gap and turn on or wake up the microcontroller for full operation. After this, the microcontroller can perform various functions such as recording the electric current across the switch gap, measuring the resistance of the switch, estimating the concentration of the target chemical compound, sending electronic signals to a display or wireless transmitter, and others. In some examples, the microcontroller can go back to sleep mode if the concentration of the target chemical compound falls back below the threshold concentration. It is noted that even when the microcontroller is in “sleep mode” the digital chemical analyzer can still continuously monitor for the presence of the target chemical compound. The positive and negative electrodes can have a continuous voltage applied during sleep mode. Thus, the wake-up circuit can be triggered any time the switch gap is bridged by molecules of the target chemical compound. The digital chemical analyzer can consume little or no power while in sleep mode until the switch gap is bridged.

The threshold concentration of a target chemical compound that will switch the chemically selective percolation from the “off” state to the “on” state can depend on the specific design of the switch and the binding properties of the binding agent, if used. In some examples, the threshold concentration can be from 1 part per billion (ppb) to 1,000 parts per million (ppm). In further examples, the threshold concentration can be from 10 ppb to 100 ppm, or from 100 ppb to 100 ppm, or from 1 ppm to 100 ppm, or from 10 ppm to 100 ppm, or from 10 ppb to 1 ppm, or from 100 ppb to 1 ppm, or from 10 ppb to 100 ppb.

### Examples

Two different 3D percolation array-based sensors (digital chemical analyzers) were fabricated having dimensions of 16×16×2 and 25×25×2 (the numbers of binding sites in the longitudinal, lateral, and vertical directions, respectively). The sensors were capable of operating at about 4 nW power by utilizing a wake-up function. The operating voltage was 2.7 V, which was less than the 5 V operating voltage of a similar sensor that was fabricated using a 2D percolation array. These sensors were found to be capable of detecting hexanal, a volatile organic compound.

The sensors were programmed to wake up when the concentration of hexanal (the target chemical compound) was above a percolation threshold. Statistically, it is proven that the two electrodes of the chemically selective percolation switch can be connected only when the number of captured target molecules exceeds a particular threshold value. These captured molecules can form bridges in both horizontal and vertical directions, allowing 3D electrical conducting paths. When a complete path is created between the two electrodes, the electrical current flows from one electrode to the other electrode, eventually waking up the rest of the electronics. Thus, this structure remains dormant in the absence of the target compound and starts consuming power only when the target is present.

The example 3D array-based gas sensors included two distinctive designs of 16×16×2 and 25×25×2 binding sites. The design utilized square-shaped horizontal plates arranged in a periodic arrangement with layers of square plates that were offset so that corners of the plates overlapped. The 16×16×2 array had the design shown in FIG. 3. The 25/25×2 array had square plates organized in the same way, but with a larger number of plates to provide 25 binding sites in the longitudinal and lateral directions. The array structure was formed by stacking three gold layers with an average thickness of 200 nm. Each gold layer included an array of square-shaped (200×200 μm²) horizontal plates. Between the layers of plates, a thin layer of SiO₂ (˜4.0 nm) and chromium (1˜1.3 nm) was deposited and later etched away to produce nanogaps. The SiO₂ layer acted as dielectric insulation, and the Cr layer served as an adhesion layer for the upper gold electrode. After the sacrificial layers (SiO₂ and Cr) were etched, the nanogaps were manifested on the edge at the intersection of the plates at different heights. The process was similar to the process shown in FIGS. 5A-5F.

In more detail, the structure was formed by stacking each gold layer with sacrificial SiO₂ and Cr layers. First, ˜500 nm of SiO₂ was deposited on a 4-inch Si wafer in a furnace at 1050° C., which provided electrical and thermal isolation of the features from the substrate. The first layer of gold was then sputtered with a chromium layer (˜25 nm thickness) for adhesion. The gold layer was patterned using wet lithography to form first layer square plates of ˜200 nm thickness. For lithography, S1813 was used as a photoresist, and AZ300 MIF was applied as the developer. Gold wet etchant was used from Trasene. The gold etching rate was 3 nm/second.

A 4-nm thick coating of SiO₂ was deposited on these gold plates by atomic layer deposition (ALD). 3DMAS (tris(dimethylamine)silane) was used as a SiO₂ deposition precursor in ALD. Then again, the second layer of gold plates was formed with a 1.2-nm Cr layer for adhesion formed by sputtering at 15 watts of power. Due to the shallow thickness, the Cr layer was sputtered with low power to ensure uniformity of deposition throughout the layer. The third layer of gold square-shaped plates were formed using a similar process. After completing all deposition processes, the ultra-thin Cr layer was wet etched using diluted (1:10) Cr-etchant. SiO₂ was then etched by RIE (Reactive Ion Etching) to release the nanogaps from the borders of the overlapping micro-islands. For the RIE process, SF₆ gas was used. For fabrication of a 2D percolation array-based sensor, only two layers of gold electrodes were deposited, and then SiO₂/Cr layer was sandwiched between the two gold layers.

Fabricated sensors were first coated with molecular probes for functionalizing the gold electrodes of nanogaps. The fabricated sensor structures were immersed in the linker solution for 36 hours to functionalize the fabricated sensors and then cleaned with a DMF (Dimethylformamide) solution and acetone for 5 min and 2 min, respectively. In this chemical sensor, the binding agent included a thiol-functional group to bind to the aldehyde group of hexanal, the target chemical compound.

The sensors were tested by exposing the sensors to various concentrations of hexanal. Hexanal was obtained from Sigma Aldrich. The fabricated nanogap-array-based sensor was placed in a microprobe station (MPS) testing chamber (Nextron). The inlet of the testing chamber was connected to a gas mixing chamber where the dilution of pure hexanal vapors occurred by mixing with nitrogen gas. The hexanal vapor was generated by controlling the flow of N₂ in a liquid hexanal-containing flask. The flask was shielded; thus, N₂ flow generated bubbles inside the liquid hexanal. The produced bubbles were carried into a mixing chamber by a mass flow controller (MFC). Concentrations of hexanal were controlled from 43.20 ppm to 1081.34 ppm.

The following formula calculated the concentration of the target gas:

$$C ( ppm ) \frac{\frac{P \times L}{760 - {L}^{′}}}{\frac{P \times L}{760 - L} + L + {L}^{′}} \times 1 {0}^{6}$$

where L and L′ were the gas flow rates of N₂ (through the bubbler) and air, respectively; P was the vapor pressure of Hexanal (in mm of Hg) at room temperature. After each testing, the chamber was purged with N₂ for ˜2 hours.

### Electrical Measurement and Characterization:

The two electrodes of the sensor were probed with a Keithley 4200S parameter analyzer from Tektronix. Firstly, the I-V graph of each device was monitored by applying incremental voltage and measuring the current for each voltage. By observing the I-V characteristics graph, a bias voltage was chosen that allows the sensor to run at its highest sensitivity. Secondly, the selected bias voltage was applied to the sensor while the device's resistance was monitored continuously during the exposure and purging of Hexanal gas.

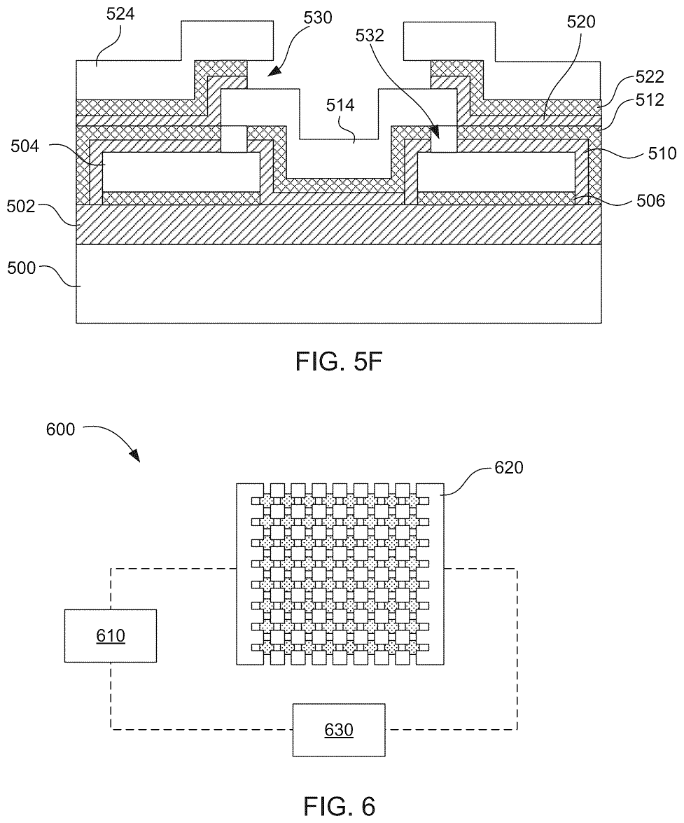

Measurement results clearly showed that the sensor structure successfully formed the metal-insulator-metal (MIM) junctions that were governed by quantum properties, showing both Direct Tunneling (DT) and Fowler-Nordheim Tunneling (FNT), as shown in FIG. 7.

Note that DT and FNT can be defined as below[11]:

$${I}_{D} ∝ V exp [ \frac{- 4 π d \sqrt{2 ϕ {m}^{*}}}{ℏ} ] \qquad ( i )$$

$${I}_{FN} ∝ {V}^{2} exp [ \frac{- 8 π d \sqrt{2 {ϕ}^{3} {m}^{*}}}{3 q ℏ V} ] \qquad ( ii )$$

Here, I_{FN} and I_{D} are the current during direct tunneling and Fowler-Nordheim tunneling, respectively. Additionally, φ is the barrier height, m* is the effective mass of electrons, V is the applied voltage, and d is the thickness of the dielectric layer. It was observed that in the FNT region, the change of current was proportional to the cube of \sqrt{ϕ}, whereas in DT region, the current was proportional to \sqrt{ϕ}. Thus, in the FNT region, the device was more responsive, as shown in FIG. 7 (bottom). However, if a MIM junction was kept deep in the FNT region for ample time, dielectric breakdown happened to cause permanent damage to the MIM junction. Thus, a bias voltage was chosen in such a way that sensor response was maximized while dielectric breakdown was prevented.

### Bias Voltage Reduction in Multi-Level Structure:

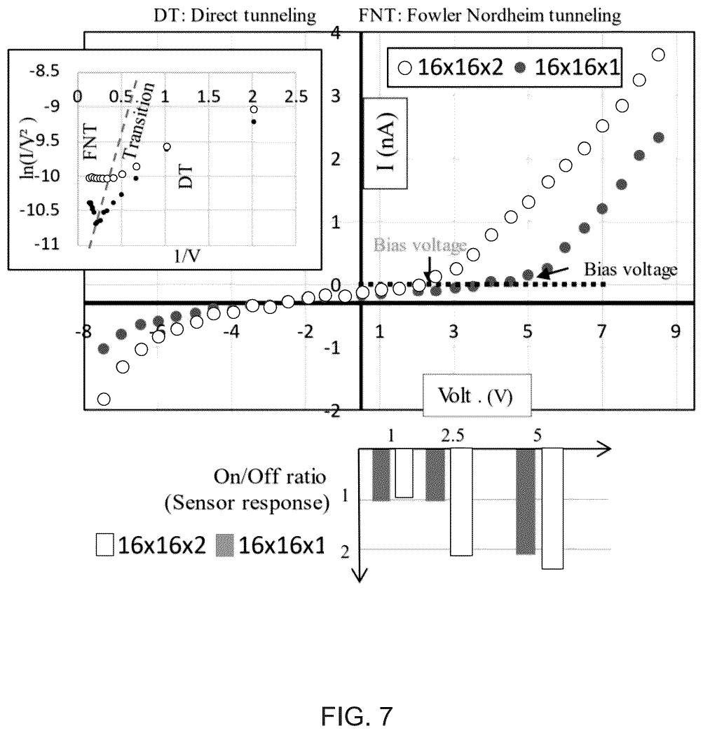

In the fabricated multi-level structure (a 3D nanogap array), the required bias voltage was reduced by 50% compared to a 2D structure. The voltage reduction was mainly due to parallel conducting paths across two layers. FIG. 8 compares the bias voltages in single layer (i.e., 2D array) and double layer (i.e., 3D) array) structures with both dimensions.

### Gas Sensing Operation:

With the exposure of 100 ppm Hexanal VOC gas, the on/off ratio response of the 16×16×2 sensor was measured at 2.38. The response was recorded for 3 continuous cycles of exposure and purging. The sensor response was defined as the following formula:

$$Response ( On / Off ratio ) = \frac{Resistance at the presenece to gas}{Resistance at the absence of gas}$$

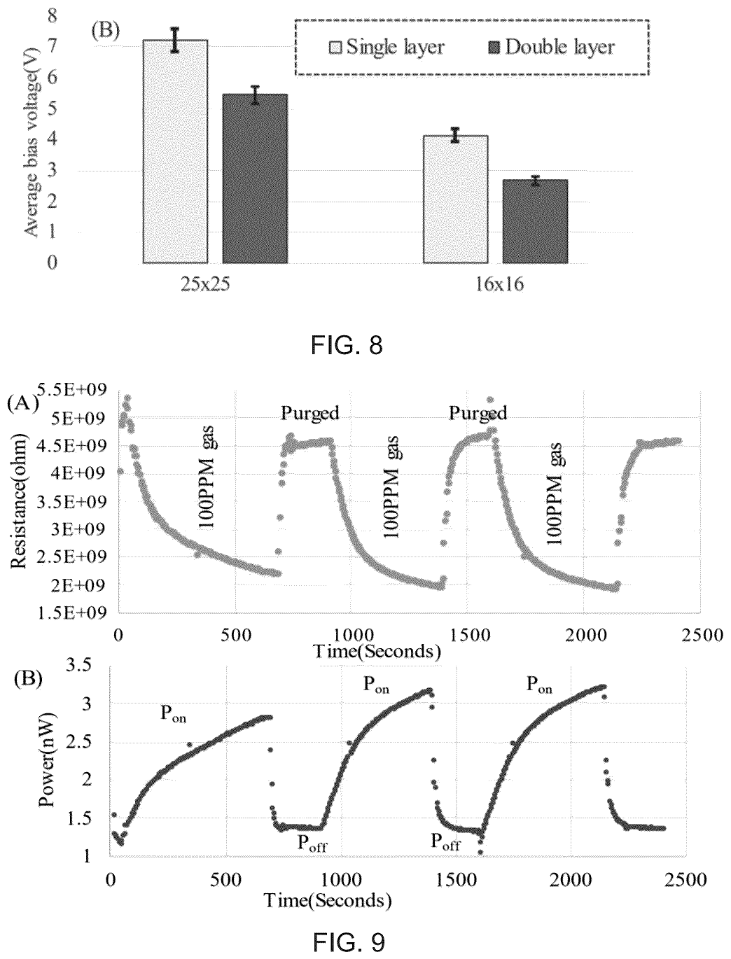

When the 16×16×2 sensor was continuously purged and exposed to Hexanal VOC, it showed similar repetitive responses, as shown in FIG. 9. The power consumption of the sensor was calculated as only 1.3 nW during the absence of target gas molecules and 3.27 nW during the presence or detection of the target gas particles. After the exposure of Hexanal VOC particles, it took nearly 500 seconds to lower the resistance to a stable saturated value; during purging, it took almost 100 seconds to return to the initial value. This behavior was almost identical when exposed to 100 ppm Hexanal gas.

In summary, the single layer sensors had a higher bias voltage of greater than 5 V, whereas the double layer sensors had a lower voltage of greater than 2.7 V. The single layer sensors had a lower probability of quantum tunneling compared to the double layer sensors. The single layer sensor used less power, at less than 1 nW, whereas the double layer sensor used 1 to 4 nW of power. However, the single layer sensor was not suitable for connecting to a 3.7 V rechargeable battery, whereas the double layer sensor was.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A three-dimensional percolation array, comprising:
a three-dimensional array of binding sites, wherein individual binding sites comprise a nanogap between electrically conductive structures, the individual binding sites being configured to selectively bind a molecule of a target chemical compound, thereby forming an electrically conductive connection between the electrically conductive structures, wherein at least a portion of the binding sites are electrically connected to at least one other binding site in a longitudinal direction, and to at least one other binding site in a lateral direction, and to at least one other binding site in a vertical direction.

2. The three-dimensional percolation array of claim 1, wherein the nanogap is sized to accommodate a single molecule of the target chemical compound.

3. The three-dimensional percolation array of claim 2, wherein the nanogap has a gap distance from 0.3 nm to 1,000 nm.

4. The three-dimensional percolation array of claim 1, wherein the binding sites comprise a binding agent attached to an electrically conductive structure on a side of the nanogap, wherein the binding agent is selective for binding to the target chemical compound.

5. The three-dimensional percolation array of claim 1, wherein at least some of the individual binding sites are electrically connected, through the electrically conductive structures, to 3 to 14 other binding sites.

6. The three-dimensional percolation array of claim 1, wherein the three-dimensional array of binding sites is made up of multiple stacked two-dimensional arrays of binding sites.

7. The three-dimensional percolation array of claim 6, wherein the two-dimensional arrays of binding sites have dimensions of 4 to 100 binding sites in a longitudinal direction by 4 to 100 binding sites in a lateral direction.

8. The three-dimensional percolation array of claim 6, wherein a number of stacked two-dimensional arrays of binding sites is from 2 to 100.

9. A chemically selective percolation switch, comprising:
a positive electrode;
a negative electrode separated from the positive electrode by a horizontal switch gap; and
a plurality of electrically conductive structures in the horizontal switch gap, wherein adjacent electrically conductive structures are separated by nanogaps forming binding sites between the adjacent electrically conductive structures, wherein the electrically conductive structures are arranged to form a three-dimensional array of binding sites having at least two stacked layers of binding sites, wherein the binding sites are distributed in the switch gap such that the binding sites are capable of binding molecules of a target chemical compound to form an electrically conductive pathway via percolation between the positive electrode and the negative electrode when the chemically selective percolation switch is exposed to a threshold concentration of the target chemical compound, and wherein the electrically conductive pathway is capable of forming in one of the layers of binding sites or in more than one of the layers of binding sites.

10. The chemically selective percolation switch of claim 9, wherein the electrically conductive structures comprise horizontal parallel plates arranges in stacked horizontal layers, wherein at least some of the horizontal parallel plates partially vertically overlap at least some other horizontal parallel plates as overlapping portions, wherein the nanogaps are vertical gaps between the overlapping portions of the horizontal parallel plates.

11. The chemically selective percolation switch of claim 10, wherein at least some of the horizontal parallel plates partially overlap with 2 to 8 other horizontal parallel plates.

12. The chemically selective percolation switch of claim 10, wherein a number of stacked horizontal layers of horizontal parallel plates is from 3 to 101.

13. The chemically selective percolation switch of claim 10, wherein at least some of the stacked horizontal layers of horizontal parallel plates comprise a two-dimensional array of horizontal plates with array dimensions of 2 to 50 plates in a longitudinal direction by 2 to 50 plates in a lateral direction.

14. The chemically selective percolation switch of claim 9, wherein the nanogaps have a gap distance from 0.3 nm to 1,000 nm.

15. The chemically selective percolation switch of claim 9, wherein the electrically conductive structures have a width from 1 μm to 1 mm.

16. The chemically selective percolation switch of claim 9, further comprising a binding agent attached to an electrically conductive structure on a side of at least some of the nanogaps, wherein the binding agent is selective for binding to the target chemical compound.

17. A digital chemical analyzer, comprising:
a power supply;
a chemically selective percolation switch connected to the power supply, wherein the chemically selective percolation switch comprises:
a positive electrode,
a negative electrode separated from the positive electrode by a horizontal switch gap, wherein the power supply is configured to apply a voltage between the positive electrode and the negative electrode, and
a plurality of electrically conductive structures in the horizontal switch gap, wherein adjacent electrically conductive structures are separated by nanogaps forming binding sites between the adjacent electrically conductive structures, wherein the electrically conductive structures are arranged to form a three-dimensional array of binding sites having at least two stacked layers of binding sites, wherein the binding sites are distributed in the switch gap such that the binding sites are capable of binding molecules of a target chemical compound to form an electrically conductive pathway via percolation between the positive electrode and the negative electrode when the chemically selective percolation switch is exposed to a threshold concentration of the target chemical compound, and wherein the electrically conductive pathway is capable of forming in one of the layers of binding sites or in more than one of the layers of binding sites; and
a detection circuit connected to the chemically selective percolation switch to output a signal based on a change in at least one of resistance and current in the chemically selective percolation switch.

18. The digital chemical analyzer of claim 17, wherein the threshold concentration is from 1 part per billion (ppb) to 1,000 parts per million (ppm).

19. The digital chemical analyzer of claim 17, wherein the power supply operates at a power less than 10 nW while the target chemical compound is present at a concentration below the threshold concentration.

20. The digital chemical analyzer of claim 17, wherein the power supply operates at a voltage from about 1.4 V to about 4.2 V.
